(19) United States
(12) Patent Application Publication
(10) Pub. No.: US 2021/0321906 A1
(43) Pub. Date: Oct. 21, 2021

(54) WALK EVALUATION SYSTEM, WALK EVALUATION METHOD, AND PROGRAM, STORAGE MEDIUM, PORTABLE TERMINAL, AND SERVER THEREFOR

(71) Applicant: Kunihiro SHIINA, Tokyo (JP)

(72) Inventor: Kunihiro SHIINA, Tokyo (JP)

(21) Appl. No.: 17/271,757

(22) PCT Filed: Aug. 26, 2019
(86) PCT No.: PCT/JP2019/033382

§ 371 Date: Feb. 26, 2021

(30) Foreign Application Priority Data
Aug. 27, 2018 (JP) 2018-170060

(51) Int. Cl. A61B 5/11

(52) U.S. Cl. CPC A61B 5/112; G01C 22/006; A61B 5/1112

(57) ABSTRACT

There is provided a system for accurately assessing a person's body condition on the basis of measured values of the person's walk. The present invention is a system for evaluating a measurement target walk and the system includes: a measurement apparatus that continuously measures the measurement target walk; and an arithmetic operation apparatus that executes an arithmetic operation based on an output of the measurement apparatus, wherein the arithmetic operation apparatus: computes a walking cycle of the walk; executes extraction of a walk in a state where the walking cycle is stable; and evaluates the measurement target on the basis of a result of the extraction.

### TECHNICAL HELD

The present invention relates to a system which measures a person's walk and evaluates the person's walking ability or the person's walking condition on the basis of measured values, and is thereby useful to determine, assess, certify, and diagnose the person's body condition.

### BACKGROUND ART

Many healthcare practitioners have mentioned that a person's walk is important in the field of anti-aging to, for example, extend healthy life expectancy and prevent dementia. So, apparatuses and systems for automatically measuring the person's walk have been put to practical use. For example, the applicant of the present invention proposed a recording apparatus capable of detecting, with high accuracy, a tendency of minute changes in a walking speed (International Publication No. WO2016/043081).

This recording apparatus is characterized in that it: acquires measurement information indicating whether a walker is walking on a flat and a straight line or not, from a sensor of a portable terminal; extracts only positional information, on the basis of the measurement information, when the walker is walking on the flat and straight line among positional information; calculates the walker's walking speed on the basis of the extracted positional information; compares the calculated walking speed with the walker's walking speed in the past; and informs the walker of a change in the walking speed.

### CITATION LIST

### Patent Literature

### PTL 1: WO2016/043081

### SUMMARY OF THE INVENTION

### Problems to be Solved by the Invention

The conventional walk measurement apparatus is intended to be useful for the prevention and improvement of a person's body condition such as aging, chronic diseases, and changes in their physical condition by discovering such body condition in a much earlier stage on the basis of changes in the walking speed. However, the person's walking condition normally changes due to factors other than the walker's own ability such as external factors for the walker, including a road congestion status, weather, walking with somebody else, shopping, and sightseeing, and internal factors including the purpose of walking. So, even if the relevant person walks on the flat and the straight line, the person's inherent ability cannot be reflected in their walking speed or step width and, therefore, there is a problem such that the person's body condition cannot be assessed more accurately. Consequently, it is an object of the present invention to provide a system capable of accurately assessing a person's body condition on the basis of measured values of the person's walk.

### Means to Solve the Problems

In order to achieve the above-described object, provided according to the present invention is a system for evaluating a measurement target walk and the system includes: a measurement apparatus that continuously measures the measurement target walk; and an arithmetic operation apparatus that executes an arithmetic operation based on an output of the measurement apparatus, wherein the arithmetic operation apparatus: computes a walking cycle of the walk; executes extraction of a walk in a state where the walking cycle is stable; and evaluates the measurement target on the basis of a result of the extraction.

### Advantageous Effects of the Invention

A system capable of accurately assessing the person's body condition on the basis of measured values of the person's walk can be provided according to the present invention.

### BRIEF DESCRIPTION OF DRAWINGS

FIG. 1 is a characteristic diagram illustrating the relationship between a horizontal axis representing time (seconds) and a vertical axis representing a walking cycle (in milliseconds) by considering two steps as a cycle in daily life of the inventor;

FIG. 2 is a characteristic diagram in which stable walking in FIG. 1 is enlarged;

FIG. 3 is a block diagram of a system according to the present invention;

FIG. 4 is a functional block diagram of a portable terminal; and

FIG. 5 is a functional block diagram of a server.

### DESCRIPTION OF EMBODIMENTS

As a result of earnest examination about a person's walk, the inventor of the present invention has found that if the person's walk is divided into stable walking and unstable walking as described later, extracts measured values during the stable walking, and sets an evaluation index by, for example, using an average value of the measured values, it is possible to obtain the evaluation index indicating the walking ability of a measurement subject, regarding which the relevant value is stable in a short term. Therefore, the inventor has been brought to the fact that it is possible to determine and assess any decline in physical performance such as the person's aging, advancing age, and enfeeblement, or enhancement of the physical performance such as physical recovery and enhancement of physical fitness by evaluating the stable walking by, for example, focusing attention on any changes in the evaluation index over time.

In people's daily life, a plurality of aspects coexist in a person's walk. For example, people walk faster or slower depending on the situation, whether conscious or unconscious, according to the purpose of the walk such as shopping, sightseeing, visiting places, searching, or jogging, or external environment such as road congestion, road crossing, the existence of an accompanying person(s), acting in a group, and bad weather.

Meanwhile, in people's daily life, they frequently walk on a straight or madly curved and flat road or passage without any obstacles without any particular walking purpose other than heading for a destination in a state without the occurrence of the external environment and they walk unconsciously rhythmically or regularly at a pace suited to themselves. This is because a person inherently has a walking function which is called a central pattern generator. The inventor will refer to this walk as stable walking and, on the other hand, refer to the former walk as unstable walking.

Specifically speaking, a walk regarding which a standard deviation of, for example, a walking cycle in an arbitrary number of continuous steps is equal to or smaller than a set threshold value is defined as the stable walking; and a walk other than the above-described walk is defined as the unstable walking. A person's daily walk is divided into the stable walking and the unstable walking according to the above-described definitions, so that it is possible to generate an evaluation index indicating the walking ability of the measurement subject.

Since the unstable walking is affected by the walking purpose and the external environment, the walking condition and manner cannot be accurately derived from the person's body condition or physical performance. On the other hand, the stable walking is not affected or less affected by the walking purpose or the external environment, so that the walking condition or manner derives from the person's body condition and the physical performance. In other words, the stable walk shows changes in the person's body condition. For example, aging, advancing age, and enfeeblement appear as changes in the index for the stable walking. On the other hand, the same can be said about, for example, the physical recovery and enhancement of physical fitness. Therefore, changes in the person's body condition and physical performance can be, for example, assessed and determined by evaluating the index for the stable walking.

However, since the stable walking coexists with the unstable walking and the stable walking does not occur regularly, it is basically not easy to distinguish and extract the stable walking from the unstable walking.

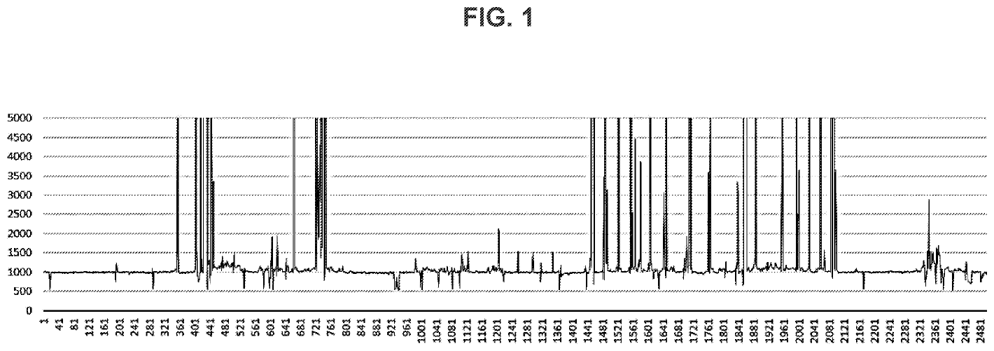

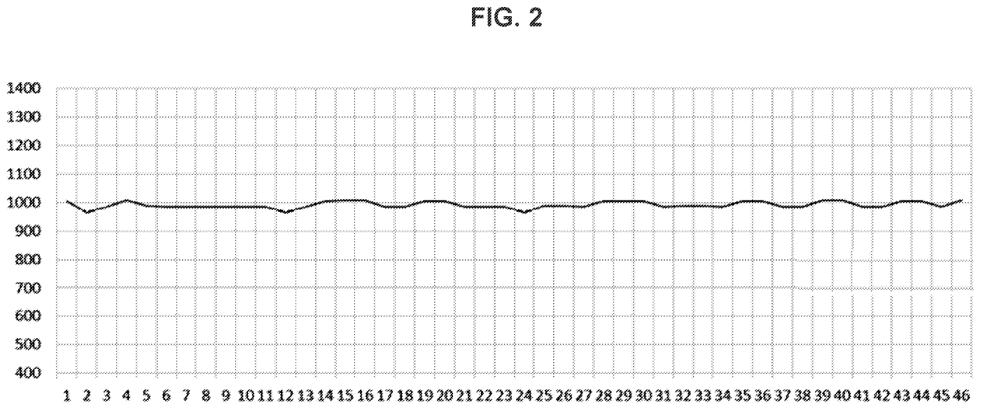

FIG. 1 is a characteristic diagram illustrating the relationship between a horizontal axis representing time (seconds) and a vertical axis representing a walking cycle (in milliseconds), each of which consists of two steps, regarding the inventor's daily life. The walk with the walking cycle of approximately 1000 milliseconds is the stable walking and the walk other than this is the unstable walking. Although the stable walking and the unstable walking coexist, you can tell that the stable walking occurs more than once during the walk. FIG. 2 is a characteristic diagram in which the stable walking in FIG. 1 is enlarged. A variation coefficient (Standard Deviation/Average Value) of the walking cycle of the stable walking is approximately 1%. The inventor of the present invention did a research on the variation coefficient of the walking cycle of healthy elderly persons' stable walking and found that the variation coefficient of the healthy elderly persons was approximately 2%.

The inventor of the present invention has defined the stable walking, which is as defined in the aforementioned definition, as unconscious autonomous walking of a person who is not conscious of trying to walk more slowly or faster and regarding which fluctuations of the walking cycle are within a predetermined threshold value. The fluctuation width may be, for example, the standard deviation or the variation coefficient. The threshold value should not be limited and may be changed according to one or more of, for example, a race, sex, age, body height, body weight, diseases, and other physical conditions. For example, the standard deviation may be within the range of 10%, preferably within the range of 5%, and more preferably within the range of 3%.

Conventionally, it has been not easy to distinguish the stable walking from the person's daily walk; however, the inventor of the present invention has decided to: find a walking cycle by continuously measuring the daily walk for a specified period of time (for examples, days, weeks, or months) by using a step count measurement function of a portable terminal (smartphone) which a person as a measurement target carries; and extract the stable walking by causing a computing function of the portable terminal and/or the server to filer the walking cycle. Then, the computing function of the portable terminal and/or the server can calculate an index for the stable walking on the basis of the walking cycle of the extracted stable walking and evaluate the person's body condition on the basis of this index. The portable terminal corresponds to a measurement apparatus in claims. The computing function of the portable terminal and/or the server corresponds to an arithmetic operation apparatus in the claims.

Since the daily walking environment and the walking purpose of each occasion vary, it is confirmed that an average value of the “walking cycle of the stable walking” which is calculated here actually fluctuates a little on a daily basis, but its fluctuation range is extremely narrow.

Since a person's “most easily walkable walking cycle” is determined according to their own characteristics including their walking ability and personality, it is assumed that their most easily walkable walking cycle does not change considerably in the short term unless they suddenly become ill; however, their most easily walkable walking cycle changes in the medium to long term due to aging or changes in physical condition. An “easily walkable step width” also changes in the medium to long term in the same manner.

On the other hand, the unstable walking excluding the stable walking is significantly affected by the external environment, so that its average walking cycle fluctuates considerably and an average value of the stable walking and an average value of the unstable walking are largely different from each other. The results have been verified and it has been confirmed that it is very meaningful to divide the walk into the stable walking and the unstable walking.

Regarding the average walking cycle during the stable walking, its fluctuation range is extremely narrow in actual measurements, so that the average walking cycle during the stable walking is considered to be a value very close to the “most easily walkable walking cycle of the measurement subject.” Specifically speaking, the person's body condition such as aging and the degree of changes in the physical condition in the medium to long term can be evaluated by following changes in the “average walking cycle during the stable walking.”

Similarly, the inventor of the present invention focuses attention on a walking ratio in order to evaluate an individual's body condition on the basis of their walking cycle. The walking ratio is a value obtained by dividing the step width by a step rate; and there are many academic research results indicating that each person's walking ratio during their free walk for a certain period of time is constant, that is, the step width is proportional to the step rate. The free walk is a walk from which the influences of the external environment are eliminated, and which is the results of measurements performed mostly on straight lines or mildly curved lines. The step rate is a value twice as much as the number of walking cycles per minute, that is, the step count per minute.

For example, an average value of the walking cycle of the stable walking which is derived from a large number of times of measurements is also considered to be that of walking under the circumstance equivalent to that of the free walk; and specifically speaking, the walking ratio in the stable walking is almost constant, that is, the walking cycle may be considered to be proportional to the step width.

The advantage of the walking ratio is that the step width can be estimated by multiplying the step rate by the walking ratio. The step rate can be easily detected by the portable terminal as described earlier. So, once the walking ratio is decided, the walking speed of the stable walking can be estimated easily and almost accurately by multiplying the step rate by the step width.

As a result of examination by the inventor of the present invention, this walking ratio was almost constant in the short to medium term. The walking ratio also gradually decreases due to, for example, advancing age. Therefore, the decrease in the walking ability caused by, for example, advancing age can be presented to the user by replacing it with a relative decrease in the walking speed during the stable walking. It is replaced with the walking speed because the walking speed is often discussed regarding superiority or inferiority in the healthy life expectancy and the walking speed is easily accepted by the user.

Furthermore, changes in the walking ratio in the medium to long term can be found by regularly measuring the step width and the walking cycle. A measurement error of the walking cycle of the stable walking is extremely small; however, it is said that in the senile generation, the degree of decrease in the step width is larger than the decrease in the step rate.

The measurement accuracy of the step width and the walking cycle is inferior to the measurement accuracy of the walking cycle of the stable walking; however, by finding minute changes in the walking cycle of the stable walking, it is possible to prompt the user to measure the step width and the walking cycle and evaluate the body condition by also considering changes in the walking ratio. In other words, it is possible to discover a sign of changes at an early stage by combining the advantages of both of them.

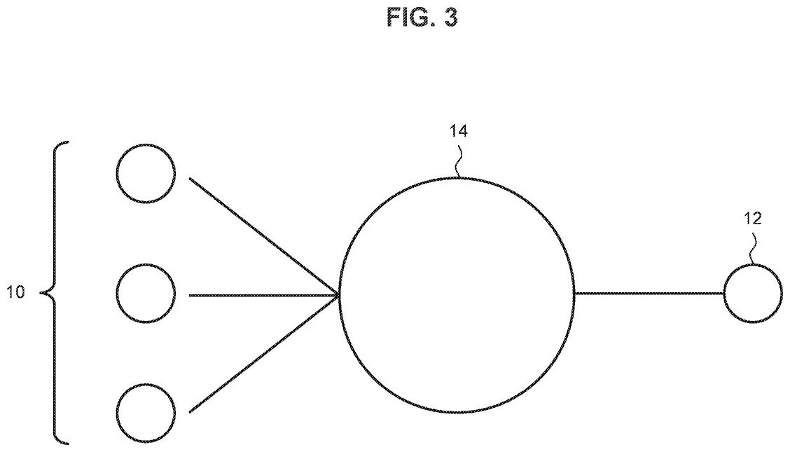

FIG. 3 is a hardware block diagram of one embodiment of a system according to the present invention. The system includes a plurality of portable terminals 10 and a server 12; and the plurality of portable terminals 10 and the server 12 are connected via a communication network 14 such as a telephone network or the Internet. The portable terminal 10 may include at least an acceleration sensor and a GPS sensor.

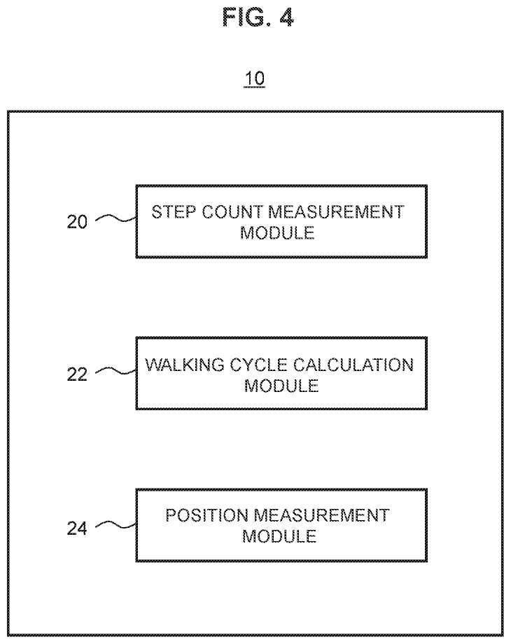

Each of the portable terminals 10 and the server 14 has a normal configuration as a calculator. The normal configuration means a controller (such as a CPU), a memory (storage medium), storage, a display, a communication unit, and so on as a computer. The storage medium may be a non-portable-type storage medium such as a hard disk drive or a flash memory drive. The portable terminal 10 may be an android smartphone, an iPhone, a portable personal computer, or a watch-type other portable equipment. The memory for the server 14 is composed of a non-portable-type storage medium such as a hard disk drive or a flash memory. FIG. 4 is one example of a functional block diagram of the portable terminal. The portable terminal 10 includes a step count measurement module 20, a walking cycle calculation module 22, and a position measurement module 24. A controller for the portable terminal 10 implements these modules by executing programs in the memory for the portable terminal 10 and then cooperating with hardware of, for example, a sensor. The modules may be paraphrased using other expressions such as means, units, circuits, blocks, units, or elements.

The step count measurement module 20 measures the step count and step count time-of-day on the basis of an output from an acceleration sensor or a software sensor such as STEP_DETECTOR or STEP_COUNTER of the android terminal (hereinafter simply referred to as the “sensor”). A method for calculating the step count and the step count time-of-day on the basis of the output from the acceleration sensor may be arbitrary.

The step count measurement module 20 continuously incorporates the output from the sensor and accumulates the step count such as the first step, the second step, and up to the nth step. The step count measurement module 20 records count time-of-day information in milliseconds of each counted number of steps (the step count information and the time-of-day information will be hereinafter collectively referred to as “step count measurement information”) in a management table. The management table exists in the memory for the portable terminal 10.

The walking cycle calculation module 22 refers to the step count measurement information in the memory and calculates a cycle of each step from the difference of the time-of-day information on the basis of the measured value of the step count and the time-of-day information of the step count measurement module 20. If all pieces of the time-of-day information indicate the count time of day of each step, their difference is the cycle of each step. Incidentally, one walking cycle is a total value of a cycle of two steps on the right and left.

However, the time-of-day information of the step count measurement module 20 does not necessarily have all the pieces of the count time-of-day information for each one step; and in that case, the count time of day for every one or two steps may be calculated by an arbitrary method on the basis of the count time-of-day information of the step count measurement module 20 and their difference may be used as walking cycle information. Incidentally, in this case as well, it has been confirmed that it is sufficiently possible to obtain the walking cycle information with the accuracy required to achieve the object of the present invention.

When a person is walking normally, the one-step cycle is 350 milliseconds or more and less than 700 milliseconds and the two-step cycle is 700 milliseconds or more and less than 1,400 milliseconds. When the one-step cycle is 350 milliseconds (171 steps/minute), it is a running state; and a cycle of 700 milliseconds (85 steps/minute) is a slow walk for which it is difficult to balance, so that regarding the above-mentioned arbitrary method, for example, it is possible to adopt an aspect of counting two steps by dividing the difference of the time-of-day information into two equal parts if the difference is 700 milliseconds or more.

The walking cycle calculation module 22 continuously records the walking cycle information from the start of the walk to its end in the management table in the memory. The management table records the time-of-day information and the walking cycle by associating them with each other with respect to each counted number of steps.

The position measurement module 24 continuously records GPS data acquisition time of day and positional information by linking the step count and the step count time-of-day information at the above-mentioned acquisition time of day to the above-mentioned information. The position measurement module consolidates such records in a table and records it in the memory.

The controller for the portable terminal 10 regularly uploads these management tables, which are recorded in the memory, to the server 12. The server 12 records the management tables for each portable terminal 10, that is, for each user in the storage.

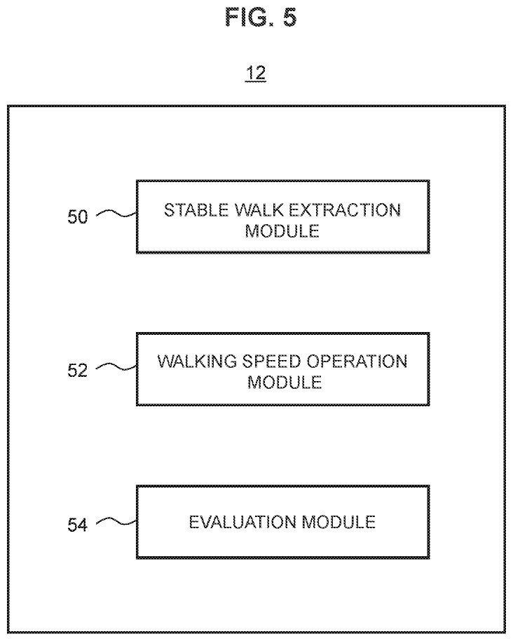

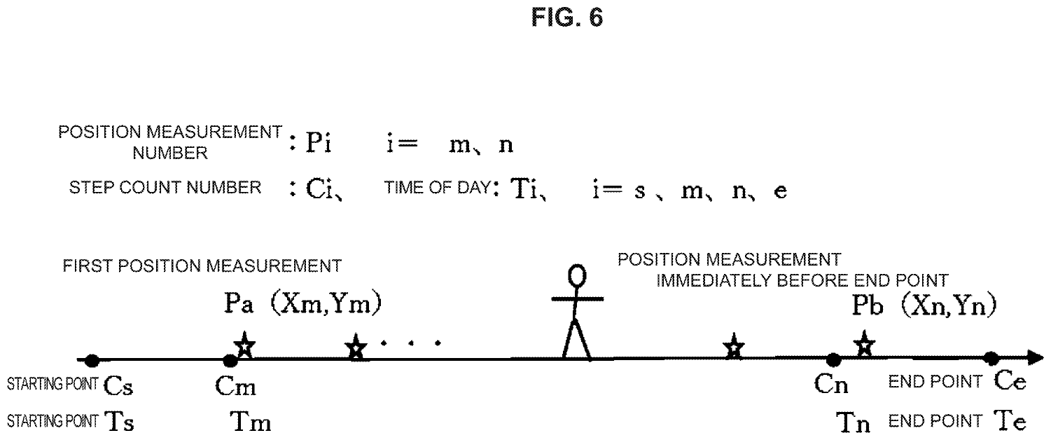

The server 12 includes, as illustrated in FIG. 5, a stable walk extraction module 50, a walking speed operation module 52, and an evaluation module 54.

The stable walk extraction module 50 extracts the stable walking for each user by referring to the management table. The stable walk extraction module 50 judges whether the walk is the stable walking or not, for example, for every 20 steps (every unit walk section). The unit walk section, in other words, a stable walking check target area is set as “20 steps” because: if the unit walk section is large, the unstable walking can be easily mixed in the stable walking; and on the other hand, if the unit walk section is small, it is difficult to distinguish the stable walking from the unstable walking. The unit walk section may be, for example, from 8 steps to 40 steps.

The stable walk extraction module 50 calculates variables (the standard deviation and the variation coefficient) of each 20-step walking cycle of the unit walk section. The stable walk extraction module 50 calculates the variables with respect to each of a plurality of unit walk sections and classifies the unit walk section(s) whose variable is equal to or smaller than a threshold value. As a result, the stable walk extraction module 50 can determine the stable walking and accumulate average walking cycle data of the stable walking in the memory.

The stable walk extraction module 50 may compare an average value of the walking cycle of each step count with an upper limit and a lower limit regarding the classified unit walk sections and exclude the unit walk section(s) which exceeds the upper limit, and the unit walk section(s) which falls under the lower limit. The upper limit is set in order to exclude fast walking or jogging (walking of a high speed range) which cannot be called normal walking; and the lower limit is set in order to exclude slow walking (walking of a slow speed range) which cannot be called normal walking.

People often walk unconsciously with the most easily walkable walking cycle and step width in their daily life. So, with the walking cycle during the stable walking, values near the walking cycle with which the relevant person can most easily walk are often and prominently observed. Therefore, the stable walk extraction module 50: finds an average value and a standard deviation of the average walking cycle data upon the stable walking, which have been accumulated within a certain period of time such as one day; finds the average walking cycle during the stable walking by excluding, for example, the fast walking and the slow walking, which occur less frequently, with respect to each user by means of a calculation process such as recalculation of the average value of only the data within a certain range from the average value; and records this in the management table with respect to each user.

The walking speed operation module 52 computes the walking speed with respect to each user. The walking speed operation module calculates the step rate from an average cycle of the stable walking and calculates the step width by multiplying this step rate by the walking ratio. The walking speed operation module 52 reviews the walking ratio for each specified time period on the basis of the calculation of the walking ratio described later in advance and stores the reviewed walking ratio in the memory. The stable walk extraction module 50 calculates the average walking cycle for each specified period of time, for example, each day, each week, each month, and every three months, and every six months, and each year and stores the calculated average walking cycle(s) in the management table.

The walking speed operation module 52 can calculate the walking speed by multiplying the step rate, which is obtained by dividing one minute by the average walking cycle/2, that is, the step count per minute by the step width obtained by multiplying the step rate, which is calculated from the average walking cycle, by the walking ratio. The walking speed operation module 52, for example, calculates the walking speed on a daily basis. Specifically speaking, the walking speed operation module 52 calculates the walking speed from the step width obtained by multiplying the step rate based on the average walking cycle for one day by the current walking ratio, and records this calculated walking speed in the management table. The walking speed operation module 52 transmits the calculated walking speed to the communication module of the portable terminal 10. The controller for the portable terminal 10 informs the user of the walking speed transmitted from the server 12 by, for example, displaying the walking speed on a display.

The walking speed operation module 52 calculates the walking ratio in advance, updates it for each specified period of time, and records it in the memory. The walking speed operation module 52 may calculate a plurality of walking ratios on the basis of the positional information, the time-of-day information of the positional information, the step count information, and the time-of-day information of the step count and determine a representative value of the walking ratio for each specified period of time in advance from the plurality of pieces of the walking ratio information. An example of an aspect where the walking speed operation module 52 calculates the walking ratio is as follows. That is a method executed by the walking speed operation module 52 regularly acquiring data of combinations of the step width and the step rate from the management table with respect to each user as described below and obtaining a relational expression between the step width and the step rate from the plurality of combinations of data. In this case, the relational expression is created to obtain an average value of the walking ratio for a certain period of time by calculating the walking ratio by dividing the step width by the step rate, or to find the walking ratio based on, for example, regression analysis.

The walking speed operation module 52 may employ the walking ratio of a model to which the user's attribute corresponds, from among models in which the walking ratios are classified into each physical attribute such as the age, sex, body height, body weight, and diseases. According to this method, the walking speed operation module 52 can continuously accumulate data of the walking ratios for each of many users and construct the models by analyzing such accumulated data. By using this model, the measurement of the step width by the portable terminal 10 becomes unnecessary.

The former method will be explained. The step width and the step rate can be calculated by the following methods,

1. Step Width (cm)=Walking Distance (m)/Step Count
2. Step Width (cm)=Walking Speed (m/minute)×Half Walking Cycle (seconds)
3. The walking cycle and the step width are calculated from walking ground touch time of day and positional information of, for example, image information.

Firstly the method 1 will be explained by using FIG. 6. Although the accuracy of positional measurement using, for example, satellite radio waves is low at present, the positional information with high accuracy can be obtained by repeatedly performing the measurement. So, the distance between a starting point and an end point can be determined from the positional information of the starting point Cs and the end point Ce.

When once the user stops at the starting point Cs and presses a “measurement start button” on the terminal, the position measurement module 24 links the step count measurement information of the relevant acquisition time of day to the GPS data acquisition time of day and the positional information and keeps recording them.

The position measurement module 24 can: determine from the operation of the “measurement start button” that the user is standing at the starting point Cs; and determine that the GPS data obtained during several tens of seconds before the user makes the next first step is the positional information of the starting point Cs, Since the time elapsed after the start of walking to the first step exceeds several seconds, the position measurement module 24 can: determine that the user is in a walking state after the first walking-start step; and gradually increase the counted number of steps which is linked to each piece of GPS data while walking.

If the user stops at the end point Ce for several tens of seconds, the time of day of the next first step will be several tens of seconds later; and, therefore, the position measurement module 24 can identify walking end time of day. If the user stops at the end point Ce for several tens of seconds, the counted number of steps at the time of acquisition of the GPS data during that time is the same. So, the position measurement module 24 determines that the GPS data with the same counted number of steps are the positional information of the end point Ce.

If the user starts walking from the end point Ce, the counted number of steps increases; and, therefore, the position measurement module 24 can determine that the measurement has been terminated. The position measurement module 24 may also terminate the measurement by using a “measurement end button” of the terminal 10.

The walk at this time is the stable walking and the walking cycle calculation module 22 calculates the average walking cycle from the step count and the step count time-of-day information during walking in the same manner as the above-described method. The walking start time of day can be estimated as the time of day which is one step before the time of day of the first walking-start step (the time of day obtained by subtracting the average walking cycle/2), so that the difference between the walking start time of day and the walking end time of day is required time of walking and the step count can be calculated by dividing the required time of walking by the average walking cycle/2.

The distance between the starting point Cs and the end point Ce is determined from the positional information of the starting point Cs and the end point Ce and the average step width is calculated by dividing this distance by the step count. The step rate is calculated from the aforementioned average walking cycle and data of a combination of the step rate and the step width is obtained.

Since the standard deviation of the positional information in a good radio wave condition is approximately 10 m, the position measurement module 24 can rapidly enhance the accuracy of the positional information by repeating the measurement. The position measurement module 24 can also correct the measured values precisely on the basis of the distance information with the enhanced accuracy.

If the position measurement module 24 performs this measured many times, the data of the combinations of the step width and the step rate increases. So, the walking speed operation module 52 for the server 12 which has received the provision of this data can create a relational expression between the step width and the step rate by means of, for example, regression analysis. If the number of samples is small, the walking speed operation module 52: may find the relational expression of the walking ratio by determining that the step width and the step rate are in a proportional relationship; or may adopt the relational expression of a model, which is classified according to the age, sex, or the like, as the walking ratio.

Next the method 2 will be explained. Let us assume that measurement coordinates immediately after starting the measurement are Pm (Xm, Ym); the measurement coordinates immediately after terminating the measurement are Pn (Xn, Yn); the measurement coordinates between them are Pi (Xi, Yi); and the positional information acquisition time of day is Ti, where i=from m to n. Assuming that the user is walking along a straight line at a constant speed, a theoretical coordinate estimation formula can be expressed as (xi=a*Ti+b, yi=c*Ti+d).

Assuming that F=Σ[(a*Ti+b−Xi)²+(c*Ti+d−Yi)²] is established where i=from m to n, a, b, c, and d are found from simultaneous equations of the least squares method of dF/da=0, dF/db=0, dF/dc=0, dF/dd=0; and the walking speed is (a²+e)^{1/2}. The walking speed operation module 54 can decide an average step width at the same time on the basis of the average walking cycle during the above-mentioned time by multiplying the walking speed by the average walking cycle/2. At the same time, the step rate is calculated from the average walking cycle. Therefore, regarding the method 2, it is not required to determine the starting point or the end point and automatic measurement at an arbitrary location becomes possible. The individual measurement coordinates include errors; however, if the number of the measurement coordinates becomes larger, the accuracy of the estimation formula is enhanced.

The position measurement module 24 may perform the measurement at a measurement walkway which is arbitrarily determined in advance, by carrying out a step width measurement mode; or if the automatic measurement is performed without specifying the walkway, the automatic measurement of the positional information is started as triggered by the extraction of the stable walking and the measurement is performed by automatically confirming that a measurement section is a straight line and the stable walking is maintained during the measurement. The position measurement module 24 divides the relevant walk section into a plurality of sections and conducts linear assessment by deriving an angle from each inner product of two vectors of the divided sections. A threshold value for that assessment may be arbitrary.

Next, the method 3 will be explained. Regarding a walkway which belongs to a building or land and is equipped with a mechanism for individual authentication or image recognition such as a terminal ID or face authentication, the measurement apparatus automatically identifies the measurement subject and measures the walking cycle and the step width from ground contact time of day and ground contact positions of sensor information and image information.

If the measurement subject is conscious of the measurement, the measurement subject tends to walk fast by being conscious of the measurement and the possibility of walking in a manner a little different from when they walk unconsciously cannot be excluded; however, the inventor has confirmed that in the actual measurement, changes in the step width according to the step rate stay within a very narrow range according to the characteristics of the measurement subject and changes in the walking ratio (the step width/the step rate) also stay within a narrow range. If the distance between the starting point and the end point of the measurement walkway is already known, that value may be input to the portable terminal 10 by, for example, a manual operation. If a plurality of users agree and use the same walkway, the portable terminal 10 can obtain the distance with high accuracy much faster by identifying the plurality of users on the basis of the coordinates of the starting point and the end point and their individual attribute information such as nicknames and sharing the distance information of the walkway.

Incidentally, since the walking speed during the stable walking is calculated by the method 2, the walking speed during the stable walking can be directly obtained without calculating the step width and the step rate. However, under the current situation where the accuracy of the positional information is low, the accuracy of the measured values is low to extract subtle changes and the measurement frequency is also low. In other words, there is a problem that a sufficient number of measurements for the analysis cannot be obtained. Furthermore, since it requires time to achieve certain accuracy after the activation of a positional information sensor such as a GPS, it is necessary to keep operating the GPS; however, this results in excessive electric power consumption. It is expected that the positional measurement accuracy will be enhanced in the near future and it becomes possible to calculate the step width with high accuracy even as a result of a small number of measurements.

The walking speed operation module 52 creates a relational expression between the step width and the step rate by using measured values of the combinations of the step width and the step rate for a plurality of number of times; there is a proportional relation between the step width and the step rate; and a constant of this proportion or a linear relation is the walking ratio. The inventor's verification has confirmed that the standard deviation of the walking ratio as a result of measurements conducted 20 times or more is less than 3% and the relational expression with quite high accuracy can be obtained. The walking ratio may be an average of the values obtained by the measurements conducted the plurality of number of times. The walking speed operation module 52 may update the relational expression, that is, the walking ratio every specified period of time (for example, every three months). Every time the walking speed operation module 52 computes and obtains the walking ratio, it registers the obtained walking ratio in the management table.

The walking speed operation module 52 reads the average walking cycle from the management table and further reads the latest walking ratio. The walking speed operation module 52 finds the step rate from the average walking cycle, calculates the step width by multiplying the step rate by the walking ratio, and then computes the walking speed. The walking speed operation module 52 computes the walking speed in one day on the basis of, for example, the average walking cycle of one day.

The evaluation module 54 detects changes in the walking speed from the management table by referring to its time history records with respect to the walking speed as the walking ability index. For example, the evaluation module 54 refers to several months of past records of the walking speed in one day and calculates a relational expression between the walking speed and the dates. For example, regarding an elderly person, a walking speed reduction ratio for the several months can be found from the relational expression. The evaluation module 54 compares this reduction ratio with a predetermined threshold value, creates an alarm display on the basis of the comparison result, and transmits this alarm display to the portable terminal 10 corresponding to the walking speed. The threshold value may be, for example, an average value of many users with respect to their age and/or sex.

If the reduction ratio is equal to or larger than the threshold value, the alarm display may be a display to encourage the user's willingness to pay attention to, or enhance, the healthy life expectancy. If the reduction ratio is smaller than the threshold value, the alarm display may be a display to praise or maintain the user's healthy life expectancy.

The evaluation module 54 may compare a deviation of the stable walking cycle or a change rate of the walking speed in the short term. For example, the short term may be several days or shorter. As an explanation of one example, people tend to think that acute brain diseases such as cerebral infarction and cerebral hemorrhage suddenly occur, but in fact some sign often occurs several days before the occurrence of the brain diseases in not a few cases. If the deviation of the stable walking cycle increases rapidly or the walking speed decreases, that person is suspected of having a risk (predictor) of an acute disease relating to the motor function or neural circuitry rather than their walking ability in the first place. The same applies to the case where their walking has become unstable due to any joint diseases or other diseases.

Furthermore, the evaluation module 54 can generate the correlation between the walking ability index of the stable walking and the body condition by means of machine learning. For example, the evaluation module 54 can: generate a polynomial expression obtained by the server via machine learning by setting falling as an objective variable and setting the walking ability index such as information of the sex, age, body weight, body height, vital data (such as a blood pressure, body fat percentage, and body temperature), and the walking speed during the stable walking (for several months) as an explanatory variable(s); and calculate the user's risk of falling down according to the polynomial expression and warn the user about this risk.

The walking ability index is not limited to the walking speed and may be fluctuations of the average walking cycle or its deviation, or fluctuations of the step width. Regarding the former example, if a threshold value determined to extract the stable walking is set to be larger than its originally set value with respect to a healthy person, most of the extracted walking cycles of the stable walking are the same as the value which was originally set as the threshold value and only the walking cycles of a “stable walking” type, which have a slightly larger fluctuation amount, are just additionally extracted. There is a high possibility that in fact the unstable walking may be mixed in the walking cycles of this “stable walking” type. However, if the walk becomes unstable, the number of the stable walking data extracted with the threshold value which is the originally set value to extract the stable walking decreases and the number of extractions including data of the stable walking type with the larger set value increases.

If the above-described tendency continues for a long period of time, it can be determined that the measurement subject's walk has become unstable; and the stable walk extraction module 50 can treat this situation as a trigger for changing the set threshold value. If the above-described situation occurs abruptly in a short period of time like several days, there is a possibility that the measurement subject's walk has abruptly become unstable, that is, there is a risk of a serious disease. Therefore, if such a phenomenon occurs, the evaluation module 54 can recognize this as the trigger and issue an alarm to the measurement subject,

If the above-described system is employed, it is possible to accurately assess a person's body condition on the basis of the measured values of the person's walk as explained above. The above-explained embodiment does not limit the present invention, but the above-explained embodiment can be changed as appropriate. For example, the possibility of the unstable walking being mixed in the extraction of the walk in the state where the walking cycle is stable cannot be denied. The above-mentioned functions of the server 12 may be consolidated in a portable terminal and the present invention may be implemented only by the portable terminal.

### INDUSTRIAL AVAILABILITY

The present invention can be utilized for a communication system configured of a smartphone and a server.

### REFERENCE SIGNS LIST

10: portable terminal
12: server
14: communication line

1. A system for evaluating a measurement target walk, comprising:
a measurement apparatus that continuously measures the measurement target walk; and
an arithmetic operation apparatus that executes an arithmetic operation based on an output of the measurement apparatus,
wherein the arithmetic operation apparatus:
computes a walking cycle of the walk;
executes extraction of a walk in a state where the walking cycle is stable; and
evaluates the measurement target on the basis of a result of the extraction.

2. The system according to claim 1,
wherein the measurement apparatus is a portable terminal which a person carries with them as the measurement target; and
wherein the arithmetic operation apparatus is a server.

3. The system according to claim 1,
wherein the arithmetic operation apparatus:
executes extraction of a walk in a state where a fluctuation range of a walking cycle of the number of a plurality of continuous steps is within a predetermined threshold value; and
evaluates the measurement target on the basis of the extracted walking cycle and its deviation.

4. The system according to claim 3,
wherein the arithmetic operation apparatus computes a walking ability index on the basis of the walking cycle and its deviation and evaluates the measurement target on the basis of a change of the walking ability index over time.

5. The system according to claim 4,
wherein the arithmetic operation apparatus computes a walking speed as the walking ability index.

6. The system according to claim 1,
wherein the measurement apparatus outputs positional information, a step count, and step count time-of-day information; and
wherein the arithmetic operation apparatus:
calculates a walking ratio on the basis of the output from the measurement apparatus;
decides a representative value of the walking ratio in advance for each specified amount of time from calculated values of a plurality of walking ratios;
executes extraction of a walk in a state where a fluctuation range of a walking cycle of the number of a plurality of continuous steps is within a predetermined threshold value;
calculates a step rate on the basis of the extracted walking cycle;
finds a step width from the step rate and the representative value of the walking ratio; and
computes a walking speed as a walking ability index on the basis of the step rate and the step width.

7. The system according to claim 1,
wherein the measurement apparatus implements continuous measurement of the measurement target walk by:
measuring a step count; and
measuring step count time-of-day information; and
wherein the arithmetic operation apparatus:
calculates a step rate on the basis of the extracted walking cycle;
computes a step width by multiplying the step rate by a walking ratio;
calculates a walking speed on the basis of the step rate; and
calculates the walking ratio in advance for each specified period of time on the basis of positional information and time-of-day information of the measurement apparatus, and step count information and the step count time-of-day information.

8. The system according to claim 7,
wherein the measurement apparatus implements continuous measurement of the measurement target walk by:
measuring the step count on the basis of output of an acceleration sensor; and
measuring the step count time-of-day information; and
wherein the arithmetic operation apparatus:
calculates the step rate on the basis of the extracted walking cycle;
computes the step width by multiplying the step rate by the walking ratio;
calculates the walking speed on the basis of the step rate; and
acquires the walking ratio by employing a model regarding which the walking ratio is classified to each attribute of a user as the measurement target.

9. The system according to claim 4,
wherein the arithmetic operation apparatus:
compares a degree of the change over time with a specified assessment threshold value;
creates a warning message if it assesses that the degree of the change over time exceeds the assessment threshold value; and
outputs the message.

10. A method of evaluating a measurement target walk, comprising:
a measurement step of continuously measuring the measurement target walk; and
an arithmetic operation step of executing an arithmetic operation on the basis of a result of the measurement,
wherein the arithmetic operation step:
computes a walking cycle of the walk;
executes extraction of a walk in a state where the walking cycle is stable; and
evaluates the measurement target on the basis of a result of the extraction,

11. A program for evaluating a measurement target walk and causing a computer to execute an arithmetic operation step based on a result of continuous measurement of the measurement target walk,
wherein the arithmetic operation step includes:
a step of calculating a walking cycle of the walk;
a step of executing extraction of a walk in a state where the walking cycle is stable; and
a step of evaluating the measurement target on the basis of a result of the extraction.

12. A computer-readable, non-portable storage medium with the program stated in claim 11 recorded therein.

13. The portable terminal according to claim 2,

14. The server according to claim 2.
